(19) United States
(12) Patent Application Publication
(10) Pub. No.: US 2021/0027878 A1
(43) Pub. Date: Jan. 28, 2021

(54) FAST AND PERSONALIZED RECOMMENDER SYSTEM FOR RADIATION THERAPY PLANNING ENHANCEMENT VIA CLOSED LOOP PHYSICIAN FEEDBACK

(71) Applicant: KONINKLIJKE PHILIPS N.V., EINDHOVEN (NL)

(72) Inventors: Ze HE, CAMBRIDGE, MA (US); Kevin LYONS, CAMBRIDGE, MA (US); Gajendra Jung KATUWAL, SOMERVILLE, MA (US); Christine Menking SWISHER, SAN DIEGO, CA (US)

(73) Assignee: KONINKLIJKE PHILIPS N.V., EINDHOVEN (NL)

(21) Appl. No.: 17/040,119

(22) PCT Filed: Mar. 14, 2019
(86) PCT No.: PCT/EP2019/056364

§ 371 Date: Sep. 22, 2020

Related U.S. Application Data
(60) Provisional application No. 62/646,957, filed on Mar. 23, 2018.

(51) Int. Cl. G16H 20/40; G16H 40/20; G16H 50/70; A61N 5/10

(52) U.S. Cl. CPC G16H 20/40; A61N 5/1038; G16H 50/70; G16H 40/20

(57) ABSTRACT

A non-transitory computer-readable medium stores a preferences database (16); instructions readable and executable by at least one electronic processor (20) to perform a proposed radiation treatment plan review process (100), including: via a reviewing graphical user interface (GUI) (28), presenting a proposed radiation treatment plan to a reviewer; via the reviewing GUI, receiving one of (i) an acceptance of the proposed radiation treatment plan or (ii) a rejection of the proposed radiation treatment plan in combination with annotations of the rejected proposed radiation treatment plan from the reviewer; and updating radiation treatment plan preferences of the reviewer stored in the preferences database based on the acceptance of the proposed radiation treatment plan or based on the annotations of the rejected proposed radiation treatment plan; and instructions readable and executable by at least one electronic processor (32) to perform a radiation treatment planning process (200) including: optimizing radiation treatment parameters for a patient with respect to dose objectives and using at least one planning image of a patient to generate one or more candidate radiation treatment plans for the patient; retrieving, from the preferences database to a planning GUI (40), radiation treatment plan preferences of a reviewer associated with the patient; and displaying the radiation treatment plan preferences of the reviewer associated with the patient at the planning GUI.

### FIELD

The following relates generally to the radiation treatment arts, radiology arts, radiation planning arts, adaptive radiation treatment plan arts, and related arts.

### BACKGROUND

Radiation treatment is planned on an individual patient basis, taking into account the specific patient anatomy, the shape, size, and possibly other characteristics of the tumor, lesion, or other malignant tissue, and therapeutic goals in order to design a radiation treatment plan that delivers targeted dosages of radiation to the tumor. Trade-offs are usually required, e.g. at a boundary between the tumor and an organ at risk (OAR), the beneficial radiation dosage to the tumor tissue must be balanced against detrimental radiation exposure to the OAR. The radiation treatment planning workflow is a cooperative effort by which a radiation physicist designs the radiation delivery device parameters and delivery sequence to substantially achieve the goals of the patient's physician.

Typically, the physician prescribes the desired dose for the region of interest. This includes annotating multiple slices of a computed tomography (CT) scan with the target dose for particular regions and classifying these regions into 2 categories: (1) targets/objectives (i.e., areas of the patient that we want to deliver a critical dosage to); and (2) organs at risk/constraint (i.e., areas of the patient where we want to minimize dosage values and subsequent soft tissue damage. This dosage is typically a range for each organ). The radiation physicist receives this prescription and must generate a therapy plan to deliver it to the patient. This step involves running a physics-based optimization to maximize the dose to the target area while minimizing the dose to surrounding soft tissues. Once the optimization is complete, many feasible solutions (sometimes can be more than 20) will be generated, and the physicist will look through them and submit one plan back to the physician. The physician either accepts or rejects the plan. If the plan is rejected, the physician will likely provide some in person or informal/undocumented writing of why the plan is rejected. This rationale for rejection is typically not stored in an organized archival data structure, and is typically not formally used for later plan generation. If the plan is rejected, the physicist will then need to restart the optimization process, adjusting parameters as appropriate based on the physician feedback. These operations are repeated until the physician eventually accepts a feasible dosage plan for the patient.

After approval of the radiation treatment plan, the patient receives radiation treatment for a fixed period of time, often a month, before a physician prescribes a new treatment. In fractionated radiation treatment, the total radiation dosage is delivered over a certain number of fractions prescribed in the plan, with each fraction being a therapeutic radiation delivery session and the fractions being spaced part in time by days or even weeks. Fractionated radiation treatment has certain benefits, such as facilitating healing between fractions of healthy tissue that is exposed to the radiation. However, the extended time frame of the fractionated radiation treatment means that changes can occur which are not accurately accounted for in the approved radiation treatment plan. For example, the tumor may shrink in size due to effective radiation therapy, internal organs can shift as the patient gains or loses weight (weight loss being common during radiation therapy), or so forth.

Adaptive planning (e.g., adaptive radiotherapy or ART) is a feature provided in some commercial radiation treatment planning software. ART enables the treatment prescription to be updated to meet the change of patient status during treatment. However, ART is underutilized in many clinical settings. Implementation of ART entails sending a current CT image of the patient back to the treatment planning system (TPS) where further physics-based optimization is performed using the updated anatomy presented in the current CT image. This time-intensive and costly procedure is difficult to justify unless there is strong evidence for the benefit of doing it. Furthermore, adjustments to the radiation treatment regimen due to other factors is generally not done. Although the physician monitors the patient over the course of the treatment, it is difficult to translate biometrics outcome measures, side effects such as bleeding, impact on appetite, and overall subjective feeling such as pain, tumour size/position change over time, and other demographics information such as age, gender, genetics, medical history, into actionable adjustments to the radiation treatment plan. Prescribing a treatment plan tailored to individual patient's need, condition, and disease progression over time is therefore a daunting task, and impose significant cognitive burden to physicians.

In addition, a plan generation takes several iterations and is very time consuming, and different physicians may have different preferences, often hospitals have limited staff and time resources to implement adaptive planning.

The following discloses new and improved systems and methods to overcome these problems.

### SUMMARY

In one disclosed aspect, a non-transitory computer-readable medium stores a preferences database; instructions readable and executable by at least one electronic processor to perform a proposed radiation treatment plan review process, including: via a reviewing graphical user interface (GUI), presenting a proposed radiation treatment plan to a reviewer; via the reviewing GUI, receiving one of (i) an acceptance of the proposed radiation treatment plan or (ii) a rejection of the proposed radiation treatment plan in combination with annotations of the rejected proposed radiation treatment plan from the reviewer; and updating radiation treatment plan preferences of the reviewer stored in the preferences database based on the acceptance of the proposed radiation treatment plan or based on the annotations of the rejected proposed radiation treatment plan; and instructions readable and executable by at least one electronic processor to perform a radiation treatment planning process including: optimizing radiation treatment parameters for a patient with respect to dose objectives and using at least one planning image of a patient to generate one or more candidate radiation treatment plans for the patient; retrieving, from the preferences database to a planning GUI, radiation treatment plan preferences of a reviewer associated with the patient; and displaying the radiation treatment plan preferences of the reviewer associated with the patient at the planning GUI.

In another disclosed aspect, a non-transitory computer-readable medium stores instructions readable and executable by at least one electronic processor to perform a radiation treatment plan and approval method. The method includes: receiving, at a first access point, a proposed radiation treatment plan from a second access point; receiving, via one or more user input devices at the first access point, one or more user inputs indicative of at least one of an acceptance of the proposed radiation treatment plan or a rejection of the proposed radiation treatment plan in combination with annotations of the proposed radiation treatment plan; transmitting the acceptance or the rejection in combination with the annotations to the second access point and displaying, at the second access point, the acceptance or the rejection in combination with the annotations; and storing the acceptance or the rejection in combination with the annotations in a preferences database.

In another disclosed aspect, an adaptive radiation planning method to perform fractionated radiation therapy on a patient over a plurality of radiation treatment sessions in accord with a radiation treatment plan. The method includes, between successive sessions of the fractionated radiation therapy: constructing a current state of the patient with state variables derived from a current medical image of the patient and additional state variables derived from patient information other than the current medical image of the patient; by a processor, applying a neural network to the current state to generate an adaptive radiotherapy (ART) recommendation; displaying the ART recommendation on a workstation and receiving a decision as to whether to perform ART via the workstation; by the processor, performing ART to adjust the radiation treatment plan conditional upon the decision being to perform ART; and by the processor, performing reinforcement learning based on the decision to update the neural network.

One advantage resides in reducing the amount of time and cost for a physician to select a proposed radiation treatment plan.

Another advantage resides in storing reasons of a physician in rejecting a proposed treatment plan and using these reasons in generation of future plans for the physician.

Another advantage resides in reducing a rejection rate of proposed treatment plans by a physician.

Another advantage resides in adaptive learning to generate proposed treatment plans more quickly and efficiently.

Another advantage resides in adaptively updating the treatment plan during implementation of the plan.

A given embodiment may provide none, one, two, more, or all of the foregoing advantages, and/or may provide other advantages as will become apparent to one of ordinary skill in the art upon reading and understanding the present disclosure.

### BRIEF DESCRIPTION OF THE DRAWINGS

The disclosure may take form in various components and arrangements of components, and in various steps and arrangements of steps. The drawings are only for purposes of illustrating the preferred embodiments and are not to be construed as limiting the disclosure.

FIG. 1 diagrammatically shows a radiation treatment planning and approval system according to one aspect; and

FIGS. 2-4 show exemplary flow chart operations of the system of FIG. 1.

### DETAILED DESCRIPTION

The following discloses approaches for customizing/improving radiation therapy planning processes. In these processes, there are at least two actors: the oncologist (or more generally, a physician), and a radiation physicist. The oncologist handles the medical side, and develops objectives for radiation dosages delivered to a tumor and for minimizing radiation dosages delivered to an organ at risk (OAR). The radiation physicist then employs a Treatment Planning System (TPS) to run simulations to determine physically realizable radiation dosage distributions that (mostly) achieve these objectives. This is done by an inverse planning process in which parameters of the linear accelerator (linac; more generally, radiation delivery device) are adjusted and the resulting dose distributions calculated. In practice, it is often impossible to identify delivery parameter values to produce a dose distribution that fully achieves all objectives. For example, there may be regions where the tumor contacts an OAR, so that some trade-off must be made between achieving the prescription dose right up to the edge of the tumor, versus not exceeding some maximum dose anywhere in the OAR. It is typical for the radiation physicist to generate several candidate radiation therapy plans that achieve the oncologist's various objectives to varying degrees, for example with some candidate plans erring on the side of fully dosing the tumor at the expense of some excess radiation exposure to an OAR; versus others erring on the side of fully protecting the OAR at the expense of reduced dose to some portion of the tumor.

The radiation physicist then selects the “best” plan (this is subjective) and proposes it to the oncologist by email or other means. The oncologist may accept the proposed plan, or may reject it. This is usually done informally, e.g. via a telephone call or in-person meeting. If the originally proposed plan is rejected then the radiation physicist goes back and performs further optimization taking into account feedback from the oncologist on the original proposal. This can result in multiple time consuming iterations of proposing a radiation plan to the oncologist, receiving rejection/feedback, further optimizing the plan, and so forth.

A further problem is that different oncologists have different preferences, and the radiation physicist typically works with numerous oncologists. Thus, the radiation physicist must learn the individual preferences of each oncologist and remember to take those preferences into account when performing radiation plan optimizations.

In some embodiments disclosed herein, a recommender system is disclosed to address these problems. The system includes a user interface via which the oncologist accepts or rejects the proposed radiation treatment plan, and for a rejected plan adds annotations identifying desired improvements. These may be entered, for example, by using the region contouring capabilities of the TPS to identify a region for which the dose distribution provided by the plan is unsatisfactory (e.g., an under-dosed edge of the tumor proximate to an OAR), and annotating the contoured region with a new objective. The acceptances and rejections, and the annotations, are stored in a physician preferences database. As this database develops, it can be referenced by the radiation physicist at the TPS to provide oncologist-specific recommendations to the radiation physicist when performing new dose optimizations. For example, given the identity of the oncologist, the system may identify past patients of that oncologist who are similar to the current patient (e.g., same type/stage/grade of cancer, demographic similarity, and so forth) and search the physician preferences database to extract the accepted plans for those similar patients along with any annotations of rejected plans for those similar patients. This information may be displayed in an oncologist-specific recommendations window for consideration by the radiation physicist when selecting which candidate radiation treatment plan to propose to the oncologist.

In a more advanced embodiment, the system may compare the acceptances/rejections and annotations of the database with the candidate plans to directly recommend one plan for proposal to the oncologist, or to produce an oncologist-specific ranking of the candidate plans.

In a further variant, the recommender system may be employed during the dose optimizations to recommend, for example, adding a region with corresponding objective(s) to the optimization criteria based on annotations on past rejected plans adding such a region.

In some embodiments disclosed herein, an improved adaptation of radiotherapy is disclosed. Adaptive radiotherapy (ART) is a capability provided with some TPS that allows for adjustment of the original radiation therapy plan based on changes to the patient over the course of a fractionated radiation therapy regimen. However, a problem arises, as follows: in many cases, there is no incentive to update the original radiation therapy regimen absent evidence that doing so will improve patient outcome. Thus, adaptive radiotherapy is only applied if a current computed tomography (CT) or magnetic resonance (MR) image shows substantial changes in the patient that make the benefit of adaptation readily evident.

In disclosed improved adaptation approaches, a state of the patient is tracked, where the state is defined by state variables which may include image features of a current CT or MR image but also include other potentially relevant information such as patient demographic information, patient weight changes, other changes in patient condition over the course of the treatment, other treatments which the patient is undergoing, medication that the patient has been prescribed, physiological conditions of the patient, or so forth. Reinforcement learning (RL) is applied, in which a neural network is trained to propose updates for the radiation treatment regimen. In RL, ideal behaviour (e.g. as represented by a policy function) is learned within a specific context (environment) by maximizing a received reward (i.e. feedback). In one example RL application, when an agent takes an action at the current state, the RL system receives an immediate reward and updates the expected long term reward, and the state gets updated. The goal of the learning process is to maximize the overall reward over time. This machine learning approach works well in cases where the search space can be very large, and the RL system can be trained sequentially (online) starting with small data size. These properties of RL makes it suitable for use in personalized healthcare applications. The RL system can be implemented using a deep reinforcement network, called a Deep Q Network (DQN), or other suitable neural network, which is trained to learn the optimal action for a given state. Embodiments disclosed herein employ RL in conjunction with a defined learning process and state data in order to apply it for recommending ART or other adjustments to be made over the course of a radiation therapy regimen.

The neural network of the RL system is trained in an ongoing adaptive fashion, based on positive or negative feedback, e.g. whether the proposed regimen update is accepted or rejected by the oncologist (or, in a more advance embodiment, based on an update rating assigned by the oncologist, e.g. between 1 and 5), or whether patient condition improves or degrades (or whether improvement/degradation accelerates/slows) after implementing the updated regimen. The feedback can be immediate (e.g. the physician accepts or rejects the update) or delayed (e.g., whether the patient condition improves or degrades over time subsequent to implementing the proposed change). The neural network should be such that it can be trained on both immediate and delayed feedback, e.g. a deep Q network.

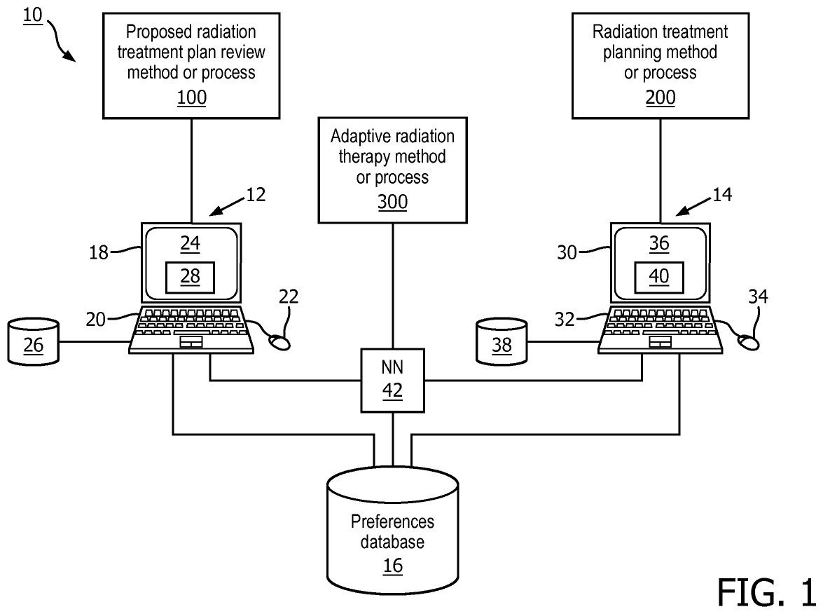

With reference to FIG. 1, an illustrative radiation treatment planning and approval system 10 is shown. As shown in FIG. 1, the system 10 includes a first access point 12 operable by a reviewer or a doctor (e.g., an oncologist, sometimes referred to herein as the doctor's workstation 12), a second access point 14 operable by a radiation physicist, and a preferences database 16 operatively connected with the first and second workstations. The first access point 12 comprises a computer, a workstation, a tablet, or other electronic data processing device 18 with typical components, such as at least one electronic processor 20, at least one user input device (e.g., a mouse, a keyboard, a trackball, and/or the like) 22, and a display device 24. It should be noted that these components can be variously distributed. For example, the electronic processor 20 may include a local processor of a workstation terminal and the processor of a server computer that is accessed by the workstation terminal. In some embodiments, the display device 24 can be a separate component from the computer 18. The doctor's workstation 12 can also include one or more databases or non-transitory storage media 26 (such as a magnetic disk, RAID, or other magnetic storage medium; a solid state drive, flash drive, electronically erasable read-only memory (EEROM) or other electronic memory; an optical disk or other optical storage; various combinations thereof; or so forth). The display device 24 is configured to display a graphical user interface (GUI) 28 including one or more fields to receive a user input from the user input device 22. In general, the oncologist is logged into the doctor's workstation 12 so that it is known that actions taken at the doctor's workstation 12 are actions by the oncologist (or, more generally, doctor). The doctor may log in using any suitable authentication process, e.g. by typing in a username/password combination, or using biometric log-in (e.g. a fingerprint reader, retina reader, et cetera), a two-step authentication log-in process, or so forth.

The system 10 also includes the second access point 14 which is operable by a radiation physicist or another reviewer associated with the patient to generate a treatment planning system (TPS) plan. Given this usual utilization context, the second access point 14 is sometimes referred to herein as the TPS access point 14. The TPS access point 14 comprises a computer, a workstation, a tablet, or other electronic data processing device 30 with typical components, such as at least one electronic processor 32, at least one user input device (e.g., a mouse, a keyboard, a trackball, and/or the like) 34, and a display device 36. In some embodiments, the display device 36 can be a separate component from the computer 30. The workstation 14 can also include one or more databases or non-transitory storage media 38 (such as a magnetic disk, RAID, or other magnetic storage medium; a solid state drive, flash drive, electronically erasable read-only memory (EEROM) or other electronic memory; an optical disk or other optical storage; various combinations thereof; or so forth). The display device 36 is configured to display a graphical user interface (GUI) 40 including one or more fields to receive a user input from the user input device 34. The doctor's workstation 12 and the TPS workstation 14 are operatively connected to the preferences database 16, for example via a wired and/or wireless hospital electronic data network, the Internet, some combination thereof, and/or so forth. The preferences database 16 is configured to store information about individual physician preferences as relates to radiation treatment plans. This information can be stored in various ways. In one approach, all candidate radiation treatment plans that are submitted to the doctor for approval or rejection are stored in the preferences database 16 along with annotations made by the doctor and associated with the stored proposed treatment plans. In another embodiment, only some portion of this information is stored in the preferences database 16, e.g. only the annotations with summary information on the proposed radiation treatment plans to which the annotations pertain. In yet another approach, the preferences database 16 may store only annotations and be linked to radiation treatment plans stored in another database, such as a Picture Archiving and Communication System (PACS) database (not shown).

The system 10 is configured to perform a proposed radiation treatment plan review method or process 100 and a radiation treatment planning process 200. These processes are linked in that the radiation treatment planning process 200 generates a proposed radiation treatment plan that is then reviewed via the proposed radiation treatment plan review method or process 100. In some embodiments, the doctor's workstation 12 is configured to perform the proposed radiation treatment plan review method 100, and the TPS workstation is configured to perform the radiation treatment planning process 200. A non-transitory storage medium stores (i) instructions which are readable and executable by the at least one electronic processor 20 of the first workstation 12 and to perform disclosed operations including performing perform the proposed radiation treatment plan review method or process 100; and (ii) instructions which are readable and executable by the at least one electronic processor 32 of the second workstation 14 and to perform disclosed operations including performing the proposed radiation treatment planning process 200. In some examples, the methods 100 and/or 200 may be performed at least in part by cloud processing.

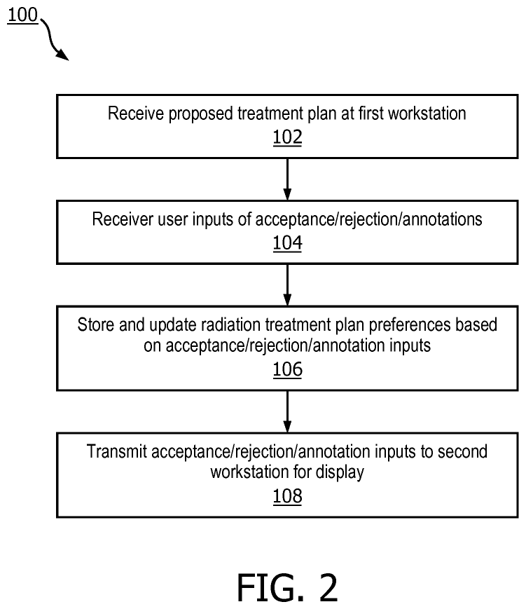

With reference to FIG. 2, an illustrative embodiment of the proposed radiation treatment plan review method 100 is diagrammatically shown as a flowchart. At 102, the at least one electronic processor 20 is programmed to control or operate the GUI 28 of the first workstation 12 to receive a proposed radiation treatment plan, for example, from the GUI 40 of the second workstation 14. For example, the doctor is logged on to the doctor's workstation 12, and the proposed treatment plan is displayed on the display device 24 thereof.

At 104, the at least one electronic processor 20 is programmed to, via the GUI 28, receive one or more user inputs indicative of (i) an acceptance of the proposed radiation treatment plan or (ii) a rejection of the proposed radiation treatment plan in combination with annotations of the rejected proposed radiation treatment plan. For example, the doctor can use the at least one user input device 22 of the first workstation 12 to input a user input to either accept the proposed radiation treatment plan, or reject the proposed radiation treatment plan and input one or more annotations indicative of changes that the doctor wishes to see to the proposed plan. In one example, the annotations can include selecting a new region of interest (ROI) for treatment, new dimensions of the ROI initially proposed in the proposed treatment plan, and the like. The selection of the ROI may in some embodiments leverage a region contouring module of the TPS (or a duplicate instance of the module at the doctor's workstation 12).

At 106, the at least one electronic processor 20 is programmed to store and update radiation treatment plan preferences of the reviewer stored in the preferences database 16 based on the acceptance of the proposed radiation treatment plan or based on the annotations of the rejected proposed radiation treatment plan. These preferences can be used to generate additional iterations of the proposed treatment plan. In addition, these preferences can be used to generate an initial future proposed radiation treatment plan.

At 108, the at least one electronic processor 20 is programmed to transmit the acceptance or the rejection in combination with the annotations to the second workstation 14. The acceptance or rejection/annotations can be displayed on the display device 36 of the second workstation 14. The operations 102-108 can be repeated for one or more subsequent proposed radiation treatment plans that are sent to the doctor for review, until the doctor accepts a proposed treatment plan.

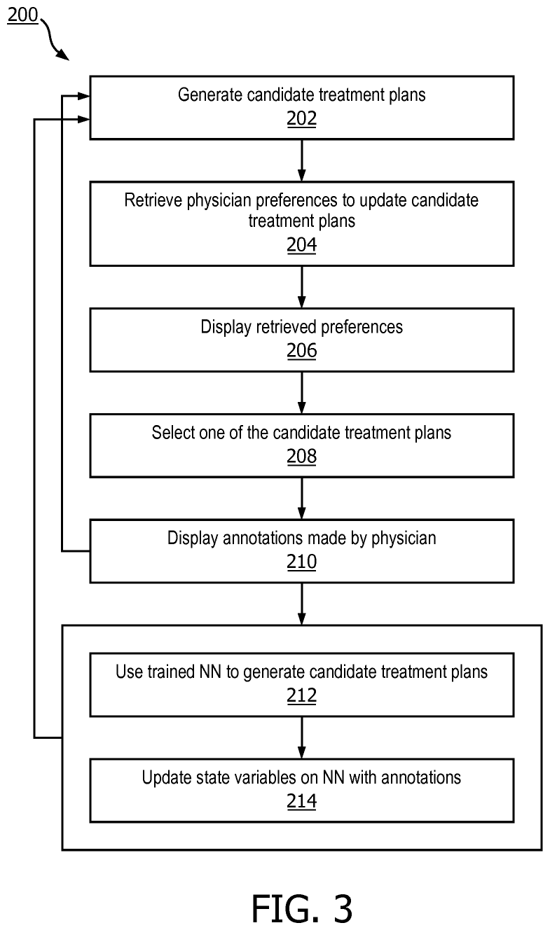

With reference to FIG. 3, an illustrative embodiment of the radiation treatment planning method 200 is diagrammatically shown as a flowchart. At 202, the at least one electronic processor 32 of the TPS workstation 14 is programmed to, via the GUI 40, generate candidate radiation treatment plans. This typically entails loading a planning image of the specific patient for whom the radiation treatment plan is being developed. The TPS workstation 14 provides a region contouring module via which the radiation physicist delineates a tumor or lesion or other radiation target along with one or more organs at risk (OARs) whose radiation exposure is to be limited. The oncologist has typically provided prescription dosages for the target and limiting dosages for the OARs. These may be variously specified, e.g. as total dosages, dose volume histogram (DVH) parameters, and/or so forth. At the TPS these are formulated as a set of objectives or goals. The radiation physicist sets up an initial radiation delivery device configuration (e.g. multileaf collimator or MLC settings, linac rotation rate, et cetera) and simulates the dose distribution that would be delivered using this configuration into the patient as represented by the planning image. The TPS computes metrics of the objectives or goals for this simulated dose distribution, adjusts the delivery device configuration and repeats the dose distribution simulation and so forth iteratively in order to optimize the radiation delivery device configuration respective to the objectives or goals. This process may be repeated a number of times, e.g. using different initial radiation delivery device configurations, different and/or differently formulated goals or objectives, or other adjustments so as to develop a set of candidate radiation treatment plans, e.g. 5 candidate treatment plans, or 10 candidate treatment plans, or 20 candidate treatment plans, or so forth. In one non-limiting illustrative example, the radiation treatment plan optimization process 202 may be implemented by the Pinnacle³ Treatment Planning System available from Koninklijke Philips N.V.

The choice of which of the candidate treatment plans generated in the operation 202 is subjective. In most cases, none of the candidate treatment plans perfectly meet all objectives or goals prescribed by the oncologist. For example, one candidate treatment plan may achieve a desired minimum dose per unit volume everywhere in the tumor, but at the cost of higher-than-prescribed dosage delivered to a portion of a neighboring OAR; whereas, another candidate treatment plan may meet the prescribed dosage in the OAR but at the cost of less-than-prescribed dose to a portion of the tumor; while other candidate plans may variously balance these two competing objectives or goals. Different oncologists may have different preferences as to the optimal way to balance these competing objectives or goals. To assist the radiation physicist in making the subjective decision as to which candidate radiation treatment plan to propose to the oncologist treating the present patient, the radiation physicist may consult the physician's preferences database 16. To do so, in an operation 204 the preferences of the reviewer associated with the patient are retrieved from the database 16 to the GUI 40, and in an operation 206 these preferences are displayed at the GUI. In an operation 208, the radiation physicist selects one of the candidate radiation treatment plans for proposal to the oncologists via the method 100 of FIG. 2. Preferably, the radiation physicist considers the oncologist's preferences displayed at 206 in making this selection. The selected candidate radiation treatment plan is then sent as the proposed radiation treatment plan to the physician's workstation 12 for acceptance or rejection/annotation by way of execution of the proposed radiation treatment plan review method 100.

At 210 (after the operations of proposed radiation treatment plan review method 100 are performed), if the proposed radiation treatment plan is rejected then the rejection is displayed at the second workstation 14 in combination with the annotations at the TPS workstation 14. Optionally, if in the operation 104 of the method 100 (FIG. 2) the oncologist contoured a new region as part of the annotation process using (an instance of) the region contouring module running at the doctor's workstation 12, then the operation 210 may include automatically importing that contour into the radiation treatment plan, with one or more objectives or goals for that added region as set forth in the oncologist's annotations. Any such addition(s) to the plan is preferably highlighted using red or another color or some other highlighting mechanism to ensure the radiation physicist is aware of these addition(s). Process flow then returns to the dose optimization process 202 but now performed for contoured regions and/or objectives or goals updated in accord with the physician's annotations.

In the retrieval step 206, the retrieved information includes radiation treatment plan preferences of the treating oncologist doctor associated with the patient. The operation 206 preferably retrieves preferences stored in the database 16 for cases similar to the present patient whose treatment is being planned. In some embodiments, information including acceptances of or annotations to radiation treatment plans of prior patients of the treating oncologist or doctor are selectively retrieved from the database 16 based on similarity to the one or more candidate radiation treatment plans for the patient generated at 202. For example, the retrieved information from the preferences database 16 can include (i) previous annotations made by an oncologist for who the proposed treatment plan is prepared; (ii) previous treatment plans accepted by the oncologist for patients having a similar ROI for treatment; and/or (iii) previous treatment plans rejected and annotated by the oncologist in which an annotation for a new ROI. The treatment plan can be updated with this retrieved information, and transmitted to the first workstation 12 for acceptance or rejection by the doctor. The preferences display operation 208 can be variously implemented. The information may be displayed in an oncologist-specific recommendations window for consideration by the radiation physicist when selecting 208 which candidate radiation treatment plan to propose to the oncologist. In another approach, the acceptances/rejections and annotations retrieved from the database at 204 may be quantitatively compared with the candidate plans generated at 202 to directly recommend one candidate plan for proposal to the oncologist, or to produce an oncologist-specific ranking of the candidate plans. The quantitative comparison may provide a quantitative assessment of each treatment plan (candidate or from the database) utilizing a metric such as a ratio comparing the extent to which goals for the tumor are met versus the extent to which goals for the OARs are met. This metric characterizes the physician's preferences as to aggressiveness, i.e. meeting the tumor goals at the expense of OARs is a more aggressive strategy compared with sacrificing tumor goals to better preserve the OARs. By comparing values of this metric for the candidate plans with values of this metric for the retrieved accepted prior plans, the recommender system can recommend the candidate plan whose aggressiveness best matches the typical aggressiveness of prior plans approved by the physician from the database 16.

In a further variant, the recommender system may be employed during the dose optimizations step 202 in order to recommend, for example, adding a region with corresponding objective(s) to the optimization criteria based on annotations on past rejected plans adding such a region. In this variant, the retrieval operation 204 must be performed during the dose optimizations 202 and the regions defined for approved prior plans are compared with the regions defined by the radiation physicist at step 202.

The step 210 in which the annotations on the proposed plan (from the method 100 of FIG. 2) are displayed can similarly employ various display approaches. A straightforward approach is to display the annotations as text in a window. As previously mentioned, if the annotations include a region newly defined by the oncologist, then the annotations may include adding this region contour with suitable highlighting. In other examples, based on the received annotations, the at least one electronic processor 32 is programmed to generate a ranked list of the candidate treatment plans from step 202 based on how well the candidate plans meet the annotation changes. (This embodiment assumes that all candidate radiation treatment plans generated at operation 202 are stored at least until after the annotations generated by the method 100 are received at the TPS workstation 14).

Referring back to FIG. 1, and within continuing reference to FIG. 3, in some examples, the system 10 can perform adaptive operations. For example, the at least one electronic processor 20 of the first workstation 12 and/or the at least one electronic processor 32 of the second workstation 14 can be programmed to apply a trained neural network (NN) 42 to recommend treatment options. In an optional operation 212, the at least one electronic processor 32 is programmed to use the trained NN 42 to recommend treatment options to generate the proposed treatment plan. The at least one electronic processor 32 is then programmed to updating the recommended treatment options using the user inputs indicative of acceptance and/or the combination of rejections and annotations.

At optional operation 214, the at least one electronic processor 32 is programmed to update one or more state variables of the trained NN 42 using the user inputs indicative of acceptance and/or the combination of rejections and annotations. The state variables can include, for example, features from imaging sessions of the patient, patient demographic information, patient weight changes, and patient condition changes.

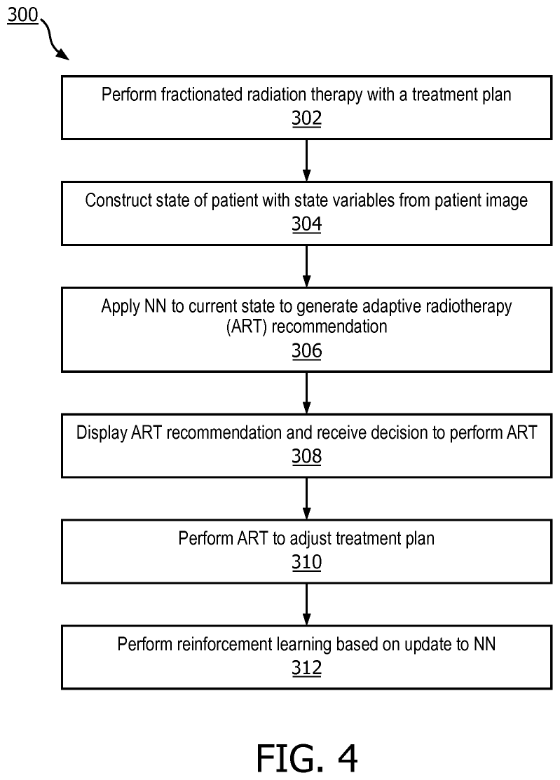

With reference to FIG. 4, an illustrative embodiment of an adaptive radiation therapy method 300 is diagrammatically shown as a flowchart. At 302, using a radiation therapy device (not shown), fractionated radiation therapy is performed on a patient over a plurality of radiation treatment sessions in accord with a radiation treatment plan. Subsequent operations 304-312 can be performed between successive sessions (i.e. successive fractions) of the fractionated radiation therapy. At 304, a current state of the patient is constructed with state variables derived from a current medical image of the patient and additional state variables including or derived from patient information other than the current medical image of the patient. In some examples, the additional state variables include or are derived from at least one of patient demographic information, patient weight changes, and patient condition changes.

At 306, with a processor 20 or 32, a neural network 42 is applied to the current state to generate an adaptive radiotherapy (ART) recommendation. In some examples, the neural network 42 comprises a Q network.

At 308, a display device 24 or 36 is configured to display the ART recommendation on a workstation 12 or 14 and receiving a decision as to whether to perform ART via the workstation. In some examples, the received decision is formulated as a received score of the ART recommendation, wherein the decision is to perform ART if the score exceeds a threshold and the reinforcement learning is performed based on the score.

At 310, with the processor 20 or 32, ART is performed to adjust the radiation treatment plan conditional upon the decision being to perform ART.

At 312, with the processor 20 or 32, reinforcement learning is performed based on the decision to update the neural network 42. In some examples, the reinforcement learning is performed further based on whether a patient condition has improved or degraded subsequent to a previous performing of ART to adjust the radiation treatment plan.

### Example 1

All previous plans and corresponding annotations, whether accepted or rejected, are stored in the preferences database 16 to be quickly queried by the system 10. The preferences database 16 acts as a library of each physician's plan history over time that can be leveraged for learning the optimal radiation treatment plan.

The annotations may also be stored in the preferences database 16. The annotations can include, for example, bounds of a ROI in 3D space (e.g., x, y, z coordinates), a correct/improved dosage range; local textual feedback, general textual feedback, a quality rating of the plan (e.g., 0-100 scale), and so forth. This annotation feedback data is then stored in the preferences database 16 to add to the library for each physician.

In embodiments disclosed herein, the annotations are used to optimize the proposed treatment plan. In some examples, a particular physician's plan library is queried for previous patients that are similar to the current one, within some threshold value of similarity. Traditionally, similarity between two patients is quantified by calculating an appropriate distance metric between the set of features representing the patients; transforming the features using different kernels before calculating the distance is not uncommon. However, estimating the patient similarity in the clinical context is a subjective task; it is very difficult to decide on the relative importance of features for similarity and the choice of kernels and distance metrics. A data-driven approach is used to quantify the patient similarity. Generative models such as variational Autoencoder (VAE) are used to create a latent space where clinically similar patients will be near to each other.

A Patient Similarity algorithm is described as follows:
a. Let X={x₁, x₂, . . . } be the set of available patients' data points
b. Train the VAE model to learn mapping function T that maps an original patient data x to a data point in a latent space x′; x′=T(x), X′={x′₁, x′₂, . . . )
c. Given a new patient x₀, map it to a latent space x′₀=T(x₀)
d. Similarity score of a patient xk to the patient x0 is the inverse of the Euclidean distance between x′k and x′₀; S(xₖ, x₀)=1/euclidean_distance(x′ₖ, x′₀); The similarity score can be other similarity metrics such as Jaccard similarity
e. Select the top similar patients based on the similarity score S

A “best in class” patient similarity algorithm is used that a physicist has control over which and the level of relevance, such as Jaccard similarity, K-Means clustering, or a ranking algorithm, that can query similar patients based on a specific set of features and the level of relevance set by physicists. There are two possible cases for this query: (1) some patients exist for this query (to be used later); or (2) no patients exist for this query, in which case a collaborative approach is used, including querying other physician libraries for previous patients that are similar to current one, or default global library.

With some set of similar patients, we extract two structures from the set: (1) the annotations for rejected patient plans, filtered to reduce dimensionality; and (2) the approved plan from the patient most similar to the current one.

The annotations are fed through a logistic regression or other machine learning algorithm to extract relevant features. The objectives and constraints of the optimization algorithm are augments with the extracted relevant features. The optimization process is initialized from previous similar optimal plans accepted for the patients listed by the order of similarity scores, from which a physicist can choose from one of them.

The optimization process is performed to generate new plans, one of which to be submitted for approval. Here a physicist can choose to stop the process when the first number N plans are generated (N can be specified by a physicist). The rationale is that for a new patient, the plan learned from the same patient or similar plan should be also accepted, and the optimization algorithm should identify those similar feasible solutions first.

### Example 2

The adaptive radiation therapy method 300 is performed using the trained NN 42. The NN 42 reads in patient state information from patient medical, physician and image database. The physician in charge is also considered as a state to generate personalized recommendations to a physician. One or more physicians agree on a set of outcome measures, such as time series disease progression outcomes (e.g., 30 day tumor size change, side effects, and overall well being rating) and normalize and assign weights to generate a reward goal. The NN 42 (e.g., a Q-network) recommends a set of actions and predicted rewards based on the state input. If a patient has started treatment, and a reward goal score is higher than previous score by a high margin, which can be determined by the physician, then the system recommends to the physician for adaptive planning review with expected outcome measures. In case of a new patient, the baseline score for comparison is not available, so, the Q-network simply recommends the action that will maximize the expected reward. A plan is chosen, generated, and implemented. A physician reviews a patient condition and provides feedback/update to the database. A patient may also have image and other physiological readings. The feedback and patient generated medical images and readings become new states, and the probability matrix of outcomes from the action is updated. These operations are repeated until a treatment plan is generated.

The NN 42 learns from two different types of feedback: 1) immediate feedback such as acceptance/rejection of the recommended adaptive planning and quality rating feedback and 2) delayed feedback such as the change in patient status over time. In the immediate learning process, inputs, such as a plan acceptance (e.g., 0/1 binary indicator, 0=denied, 1=accepted; global quality rating and feedback; and so forth) are input to the NN 42. The NN 42 is trained with standard back-propagation to minimize the loss between a current plan and the one suggested by the physician; for example, mean squared error loss between prediction and target dosage values. The NN 42 then predicts the quality of a new proposed plan and the likelihood of it being accepted by the physician. The delayed learning process occurs after the final accepted treatment plan is deliver. Inputs, such as patient survey of side effects/general feeling, a new CT image of target ROI, vital signs of the patient, and other patient outcome members are input to the NN 42. The NN 42 is trained to update conditional transition matrices from the current patient states to possible next states. The NN 42 then updates the database 16 for future plan generation.

The disclosure has been described with reference to the preferred embodiments. Modifications and alterations may occur to others upon reading and understanding the preceding detailed description. It is intended that the invention be construed as including all such modifications and alterations insofar as they come within the scope of the appended claims or the equivalents thereof.

1. A non-transitory computer-readable medium storing:
a preferences database;
instructions readable and executable by at least one electronic processor to perform a proposed radiation treatment plan review process, including:
via a reviewing graphical user interface (GUI), presenting a proposed radiation treatment plan to a reviewer;
via the reviewing GUI, receiving one of (i) an acceptance of the proposed radiation treatment plan or (ii) a rejection of the proposed radiation treatment plan in combination with annotations of the rejected proposed radiation treatment plan from the reviewer; and
updating radiation treatment plan preferences of the reviewer stored in the preferences database based on the acceptance of the proposed radiation treatment plan or based on the annotations of the rejected proposed radiation treatment plan; and
instructions readable and executable by at least one electronic processor to perform a radiation treatment planning process including:
optimizing radiation treatment parameters for a patient with respect to dose objectives and using at least one planning image of a patient to generate one or more candidate radiation treatment plans for the patient;
retrieving, from the preferences database to a planning GUI, radiation treatment plan preferences of a reviewer associated with the patient; and
displaying the radiation treatment plan preferences of the reviewer associated with the patient at the planning GUI.

2. The non-transitory computer-readable medium of claim 1 wherein the radiation treatment planning process further includes receiving, at the planning GUI, a selection of one of the one or more candidate radiation treatment plans for proposal to the reviewer associated with the patient by the proposed radiation treatment plan review process.

3. The non-transitory computer-readable medium of claim 1 wherein the displaying of the radiation treatment plan preferences of the reviewer associated with the patient at the planning GUI includes displaying the radiation treatment plan preferences as one or more recommended modifications to the one or more candidate radiation treatment plans for the patient.

4. The non-transitory computer-readable medium of claim 1 wherein the displaying of the radiation treatment plan preferences of the reviewer associated with the patient at the planning GUI includes:
comparing the one or more candidate radiation treatment plans for the patient with the radiation treatment plan preferences of the reviewer associated with the patient; and
based on the comparison, displaying a recommendation of one of the one or more candidate radiation treatment plans for the patient as most closely matching the radiation treatment plan preferences of the reviewer associated with the patient.

5. The non-transitory computer-readable medium of claim 1 wherein the retrieving, from the preferences database to the planning GUI, of the radiation treatment plan preferences of the reviewer associated with the patient includes selecting acceptances of or annotations to radiation treatment plans of prior patients of the reviewer associated with the patient based on similarity to the one or more candidate radiation treatment plans for the patient and retrieving preferences of the reviewer associated with the patient respective to the selected acceptances or annotations.

6. The non-transitory computer-readable medium of claim 1, wherein the optimizing includes:
querying the preferences database for a physician's plan library for previous patients similar to the patient;
processing the plan library to query similar patients based on a specific set of features and a level of relevance;
extracting annotations for rejected patient plans and approved plans from previous patients most similar to the patient.

7. A non-transitory computer-readable medium storing instructions readable and executable by at least one electronic processor to perform a radiation treatment plan and approval method, the method comprising:
receiving, at a first access point, a proposed radiation treatment plan from a second access point;
receiving, via one or more user input devices at the first access point, one or more user inputs indicative of at least one of an acceptance of the proposed radiation treatment plan or a rejection of the proposed radiation treatment plan in combination with annotations of the proposed radiation treatment plan;
transmitting the acceptance or the rejection in combination with the annotations to the second access point and displaying, at the second access point, the acceptance or the rejection in combination with the annotations; and
storing the acceptance or the rejection in combination with the annotations in a preferences database.

8. The non-transitory computer-readable medium of claim 7, wherein the method further includes:
generating the proposed radiation treatment plan at the second access point;
conditional upon the proposed radiation treatment plan being rejected and displaying, at the second access point, the rejection in combination with the annotations, updating the proposed radiation treatment plan based on the annotations and repeating the receiving operations, the transmitting operation, and the storing operation until a user input indicative of an acceptance of the treatment plan is received at the second access point.

9. The non-transitory computer-readable medium of claim 8, wherein the method further includes:
retrieving, from the preferences database, previous annotations made by an oncologist for who the proposed treatment plan is prepared; and
updating, at the second access point, the proposed treatment plan with the retrieved previous annotations.

10. The non-transitory computer-readable medium of claim 9, wherein the retrieving includes:
retrieving, from the preferences database, previous treatment plans accepted by the oncologist for patients having a similar region of interest (ROI) for treatment; and
updating, at the second access point, the proposed treatment plan with the retrieved previous accepted treatment plans.

11. The non-transitory computer-readable medium of claim 8, wherein the method further includes:
retrieving, from the preferences database, previous treatment plans rejected and annotated by the oncologist in which an annotation for a new region of internet (ROI) was added;
updating, at the second access point, the proposed treatment plan using the retrieved annotations.

12. The non-transitory computer-readable medium of claim 9, wherein the method further includes:
generating, at the second access point, a ranked list of the proposed treatment plans based on the retrieved previous annotations and accepted treatment plans; and
transmitting the ranked list of proposed treatment plans to the first access point.

13. The non-transitory computer-readable medium of claim 7, wherein the method further comprises:
using a trained neural network (NN) to recommend treatment options to generate the proposed treatment plan; and
updating the recommended treatment options using the user inputs indicative of acceptance, rejections, and annotations.

14. The non-transitory computer-readable medium of claim 13, wherein the method further comprises:
updating one or more state variables of the trained NN using the user inputs indicative of acceptance, rejections, and annotations.

15. The non-transitory computer-readable medium of claim 14, wherein the state variables include at least one of:
features from imaging sessions of the patient, patient demographic information, patient weight changes, and patient condition changes.

16. An adaptive radiation planning method to perform fractionated radiation therapy on a patient over a plurality of radiation treatment sessions in accord with a radiation treatment plan, the method comprising, between successive sessions of the fractionated radiation therapy:
constructing a current state of the patient with state variables derived from a current medical image of the patient and additional state variables derived from patient information other than the current medical image of the patient;
by a processor, applying a neural network to the current state to generate an adaptive radiotherapy (ART) recommendation;
displaying the ART recommendation on a workstation and receiving a decision as to whether to perform ART via the workstation;
by the processor, performing ART to adjust the radiation treatment plan conditional upon the decision being to perform ART; and
by the processor, performing reinforcement learning based on the decision to update the neural network.

17. The adaptive radiation planning method of claim 16 wherein the additional state variables include or are derived from at least one of: patient demographic information, patient weight changes, and patient condition changes.

18. The adaptive radiation planning method of claim 16 wherein the received decision is formulated as a received score of the ART recommendation, wherein the decision is to perform ART if the score exceeds a threshold and the reinforcement learning is performed based on the score.

19. The adaptive radiation planning method of claim 16 wherein the reinforcement learning is performed further based on whether a patient condition has improved or degraded subsequent to a previous performing of ART to adjust the radiation treatment plan.

20. The adaptive radiation planning method of claim 16, wherein the neural network comprises a Q network.
